(19) United States
(12) Patent Application Publication
(10) Pub. No.: US 2015/0133860 A1
(43) Pub. Date: May 14, 2015

(54) Needle Retractable-Type Replaceable-Needle Safe Self-Destructing Syringe

(71) Applicant: Zuyang WANG, (US)

(72) Inventor: Zuyang Wang, Shanghai (CN)

(21) Appl. No.: 14/381,908

(22) PCT Filed: Feb. 26, 2013
(86) PCT No.: PCT/CN2013/000190

§ 371 Date: Dec. 15, 2014

(30) Foreign Application Priority Data
Feb. 29, 2012 (CN) 201220070623.2

(51) Int. Cl. A61M 5/32; A61M 5/50

(52) U.S. Cl. CPC A61M 5/3221; A61M 5/502; A61M 2005/3206; A61M 2005/3224
USPC 604/110

(57) ABSTRACT

The present invention disclosed a retractable self-destroying safety syringe with replaceable needle including a barrel body, a needle base body (3) disposed in a barrel neck (2) at an end of the barrel body (1), an elastic buffering engaging mechanism formed between the barrel neck (2) and the needle base body (3). An asymmetrical elastic retracting locking member having an elastic function is disposed at an end of a plunger, with which an internal sleeved retracting locking member of the needle base body can be interlocked by the elastic function. After being retracted into the barrel body (1), the needle base body is deflected to one side under an asymmetric force, and the needle therefore inclines in a direction nearly a 45 degree angle to one side of the inner wall of the barrel body (1) and is blocked by the barrel neck of the barrel body, so that the needle and the needle base could not be pushed out of the barrel neck again and self-destruction of the syringe is truly achieved. The asymmetrical elastic retracting locking member is a hook (12) made of plastic material, which is formed with the plunger body by one-time molding process.

### BACKGROUND OF THE INVENTION

1. Field of the Invention

The present invention relates to a technical field of medical devices, more particularly to a retractable self-destroying safety syringe with replaceable needle, and especially to the mechanical connection structure thereof.

2. Description of Related Art

Reference Document 1, with Patent No.: ZL200710047077.4, by Application Date: 2007 Oct. 16, with Issued No.: CN101411908, the patentee thereof: Shanghai Dingjiantang Biochemical Technology Co., Ltd, has disclosed a disposable safety vaccine syringe comprising a barrel body, a plunger disposed in the barrel body, a rubber piston disposed near an end of the plunger, a needle disposed in a barrel neck of the barrel body, and a needle disposed on the needle base, which is characterized in that: a lateral notch is formed on a side wall of the end of a plunger body, which is interconnected with a vertical notch formed at the end of the plunger body of the plunger and coupled with the vertical notch; an upper part and a lower part of the needle base are sleeved together to form the needle base, wherein the lower needle base is cylindrical and has a hollow cavity and a lower needle base rib is disposed on an inner wall of the lower needle base and located near an opening of the end of the lower needle base opposite the end at which the upper needle base is disposed; an elastic member formed by interconnecting the lateral notch with the vertical notch is disposed at the end of the plunger where the plunger body has a specific elasticity, wherein the vertical notch is narrow at the top and wide at the bottom while the outer wall of the plunder body above the outer opening of the lateral notch protrudes beyond an outer wall of the plunger body therebelow, the outer opening of the lateral notch is high and the inner opening thereof is low, and the end of the plunger is in a curved shape; the upper needle base is cylindrical, wherein a plurality of reinforcing fins is disposed on the outer wall of the upper portion of the upper needle base and a flange ring of the upper needle base is disposed on the outer wall of the lower portion of the upper needle base, and the outer diameter of the flange ring of the upper needle base is greater than the diameter of the outer wall of the upper needle base by 0.05 mm to 0.3 mm; the lower needle base is cylindrical and has a hollow cavity, wherein an annular locking groove of the lower needle base is disposed on the inner wall of the cavity of the lower needle base and is coupled with the flange ring of the upper needle base, the inner diameter of the cavity above the annular locking groove of the lower needle base is less than the outer diameter of the outer wall of the plunger body above the flange ring of the upper needle base by 0.1 mm to 0.35 mm while the inner diameter of the cavity below the annular locking groove of the lower needle base is less than or equals to the outer diameter of the outer wall of the plunger body below the flange ring of the upper needle base, and an annular rib of the lower needle base is disposed on the outer wall of an end of the lower needle base opposite the end of the lower needle base rib; an upper stopping ring of the barrel neck and a lower stopping ring of the barrel neck are respectively disposed on the inner wall of the barrel neck of the barrel body and the distance between the upper stopping ring of the barrel neck and the lower stopping ring of the barrel neck is ranged from 0.3 mm to 1.5 mm, wherein the upper stopping ring of the barrel neck protrudes beyond the inner wall between the upper stopping ring of the barrel neck and the lower stopping ring of the barrel neck by 0.1 mm to 0.6 mm, the lower stopping ring of the barrel neck protrudes beyond the inner wall between the upper stopping ring of the barrel neck and the lower stopping ring of the barrel neck by 0.02 mm to 0.25 mm, the inner diameter of the barrel cavity above the upper stopping ring of the barrel neck is greater than or equals to an inner diameter of the barrel cavity between the upper stopping ring of the barrel neck and the lower stopping ring of the barrel neck while the inner diameter of the barrel cavity between the upper stopping ring of the barrel neck and the lower stopping ring of the barrel neck is less than or equals to the inner diameter of the barrel cavity below the lower stopping ring of the barrel neck, and the inner diameter of the barrel cavity between the upper stopping ring of the barrel neck and the lower stopping ring of the barrel neck is less than the outer diameter of the annular rib of the lower needle base by 0.1 mm to 0.5 mm; a stopping rib of the barrel body is disposed on the inner wall of the barrel cavity at an end opposite the end of the barrel neck and can be a linear annular rib, an annular rib consisting of several vertical ribs, a vertical rib, or an annular rib consisting of multiple protruding points; and an inner rib of the barrel body is disposed on the inner wall of the barrel body below the stopping rib of the barrel body, wherein the distance between the stopping rib of the barrel body and the inner rib of the barrel body is greater than or equals to the thickness of the annular pre-stopper piece and the inner diameter of the stopping rib of the barrel body is smaller than the diameter of the annular pre-stopper piece.

Reference Document 2, with Patent No.: ZL200720067715.4, by Application Date: 2007 Mar. 8, with Issued No.: CN201076648, the patentee thereof: Shanghai Dingjiantang Biochemical Technology Co., Ltd., has disclosed “a plunger with a metal hook” comprising a plunger body, a columnar locking member disposed on the upper end of the plunger body, and a rubber piston sleeved with the columnar locking member, which is characterized in that a mounting hole with a hook inside is disposed on the end of the plunger body at the position corresponding to the rubber piston.

Current safety syringes bear a function of retracting the needle by the hook to realize safe injections. The hook is generally made of metal pieces or plastic pieces. The metal piece requires manufacture process so as to match or be assembled on the plunger. The manufacturing process is complicated and difficult plus uncertainty of stability. Not only are the manufacturing process and cost increased, but the metal piece itself and the assembly process thereof will increase the uncertainty of risks in respect of product safety and quality. The current plastic hook still has structural defects or deficiencies, thereby leading to safety risks or vulnerabilities during use. Though the design of current plastic locking can fulfill the needle-retracting function, the needle still remains on the regular track after being retracted during use, so that it is possible to push out the needle again, which poses a risk of secondary injuries to the user.

In addition, a common problem existing in both current retractable disposable safety syringes with a single needle base and current safety syringes is how to stabilize the locking stability between an inner needle base and a barrel body to the extent satisfying stability requirements, meanwhile for the convenient use purpose, the pulling force for retracting the needle base should not be excessive. Although the rigid locking structures can achieve a stable locking effect between the inner needle base and the inner wall of the barrel body, it is hard or even impossible to meet the requirement for convenient use at the same time. The instability of plastic materials and the tendency to be easily affected by environmental temperature may lead to poor stability of the products with rigid locking structures, which makes it quite difficult to control the quality and precision during production processes and makes it impossible to realize mass production.

As mentioned above, it is still necessary to further improve the structure of the plunger and the locking structure between the inner needle base and the barrel body of the current retractable syringe, thereby further improving safety and stability during use, and achieving the purposes of simplified manufacturing process and reduced manufacturing cost.

### BRIEF SUMMARY OF THE INVENTION

The object of the present invention is to provide retractable self-destroying safety syringe with replaceable needle. An inner needle base inside the syringe and a barrel body of the syringe are interlocked with each other by an elastic buffering engaging mechanism. The locking and releasing effects therebetween are perfectly achieved by skillfully utilizing the buffering gap at the locking position and the plasticity of the locking parts. The elastic buffering engaging mechanism can be formed either on the inner needle base or on the inner wall of the barrel body, meanwhile, an corresponding engagement structure is formed either on the inner wall of the barrel body or on the inner needle base. An end of the plunger body is provided with an asymmetric locking member which has elastic functionality and can be interlocked with the cavity of the needle base of the syringe through engagement. After being retracted back into the barrel body of the syringe, the needle base is forced to deflect toward to one side under a force generated from the asymmetrical elastic mechanism, the needle therefore inclines in a direction nearly a 45 degree angle to one side of the inner wall of the barrel body and is blocked by the barrel neck of the barrel body, so that the needle and the needle base could not be pushed out of the barrel neck again. Therefore, the needle is effectively prevented from being pushed out of the barrel again which may cause secondary injuries, so that to achieve operating safety and self-destruction of the syringe, thereby to overcome and improve defects and deficiencies in the prior arts.

In order to achieve above objects, the main technical solution of the present invention is to provide a retractable self-destroying safety syringe with replaceable needle. The retractable self-destroying safety syringe with replaceable needle comprises a barrel body, a needle base body disposed in the barrel neck at the end of the barrel body, a plunger disposed in the barrel body, a rubber piston disposed near the end of the plunger. It is characterized in that: An elastic buffering engaging mechanism is formed between the barrel neck and the needle base body, which can be disposed either on an outer wall of the needle base body or on an inner wall of the barrel neck.

The present invention discloses a retractable self-destroying safety syringe with replaceable needle. The asymmetric locking member with elastic function is disposed at the end of the plunger. A combination design of the asymmetrical structure with elastic functionality can ensure that the asymmetric locking member can be smoothly inserted into the cavity of the needle base. And the interlocking between the asymmetrical locking member and a locking part of the sleeve-type retractable locking member in the needle base cavity is realized through a elastic expansion mechanism. Since the annular recess in the cavity of the sleeve-type retractable locking member also has elastic and buffering characteristic, when it contacts the asymmetric elastic locking member with being pushed, buffering and deformation correspondingly happens to both, so that the asymmetric elastic locking member can stick into the cavity of the sleeve-type retractable locking member and be interlocked with the sleeve-type retractable locking member when the asymmetric elastic locking member is being retracted. After being retracted back into the barrel body of the syringe, the needle base will be deflected to a side under a asymmetrical force, the needle therefore inclines in a direction nearly a 45 degree angle to one side of the inner wall of the barrel body and is blocked by the barrel neck of the barrel body, so that the needle and the needle base cannot be pushed out of the barrel neck again. Therefore, the needle is prevented from being pushed out of the barrel neck again which may cause secondary injuries to achieve operating safety and self-destruction of the syringe. In the embodiment of the present invention, the asymmetric elastic locking member is a plastic hook. The design of the plastic hook adopts a combination of asymmetrical and elastic structure together with applications of specific angles and sizes, and enables the plastic hook to perform both functions of buffering and interlocking. The above mentioned asymmetric elastic locking member is integrated together with the plunger body and is formed by one-time molding process, which has no need for separated manufacturing and follow-up assembly, and therefore significantly reduced manufacture costs and simplified assembly processes and procedures. The product is easy to use and functionally stable. Interlocking function between the asymmetric elastic locking member at the end of the plunger and the cavity of the inner needle base is very effective and stable. Overall and integral structure design of the product is ingenious with reasonable rationale. The product is easy to use and has wide application potentials. In comparison with designs and techniques of current products, the present invention has significant technical breakthroughs and brought the product unique safety features and practicability.

### BRIEF DESCRIPTION OF THE DRAWINGS

FIG. 1 is a schematic view of the present invention.

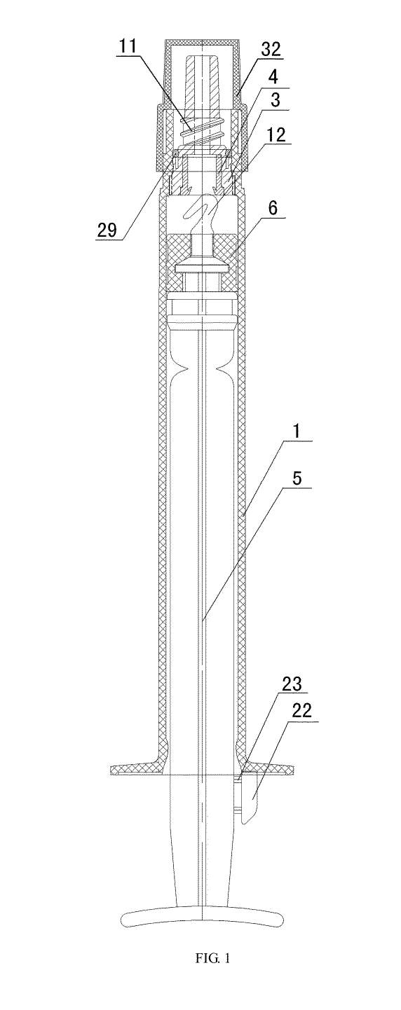

FIG. 2 is schematic diagram of a replaceable of the present invention.

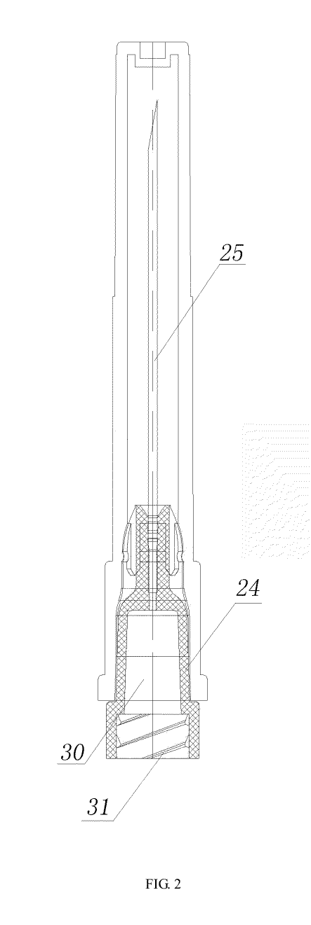

FIG. 3 is a schematic diagram of an needle base body of the present invention.

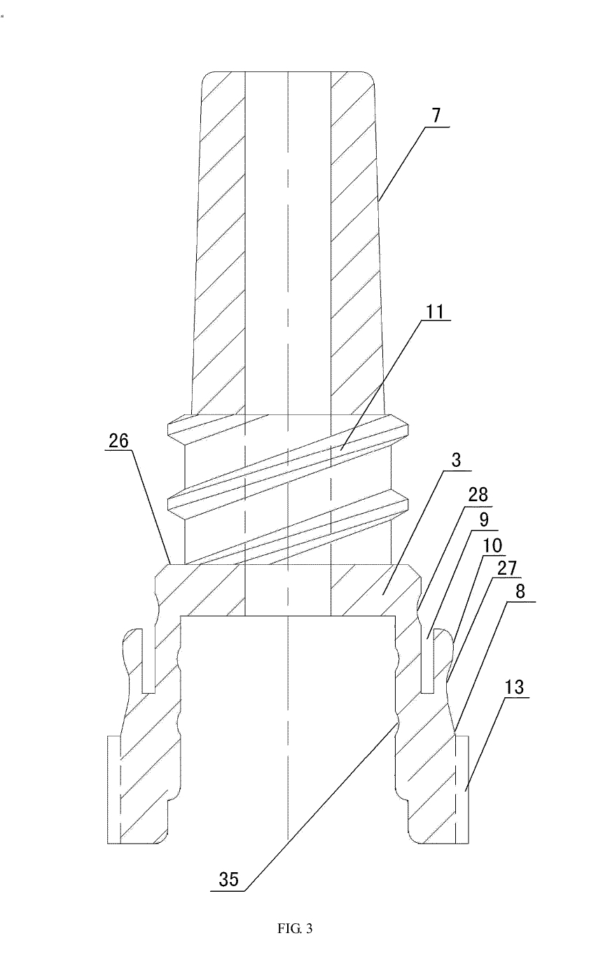

FIG. 4 is an enlarged view of FIG. 3 of the present invention.

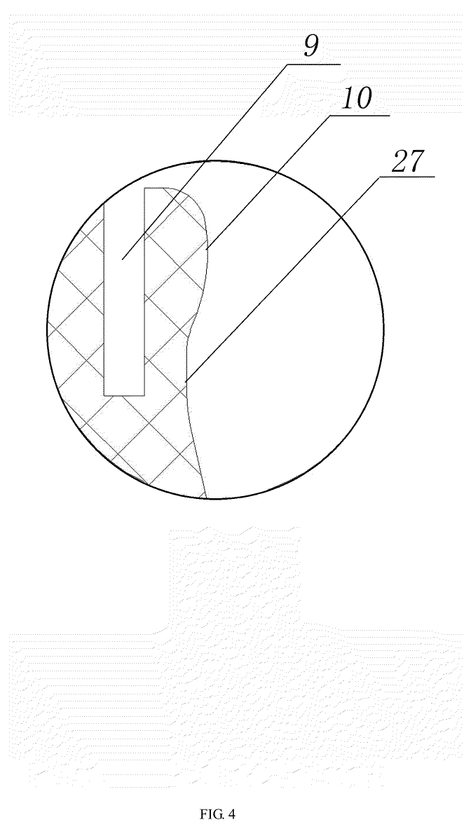

FIG. 5 is a top view of FIG. 3 of the present invention.

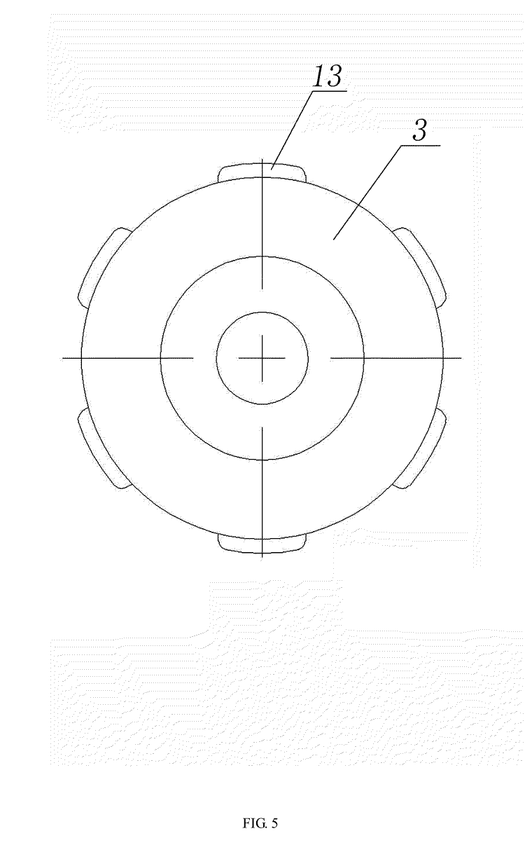

FIG. 6 is a schematic diagram of a internal sleeved retracting locking member of the present invention.

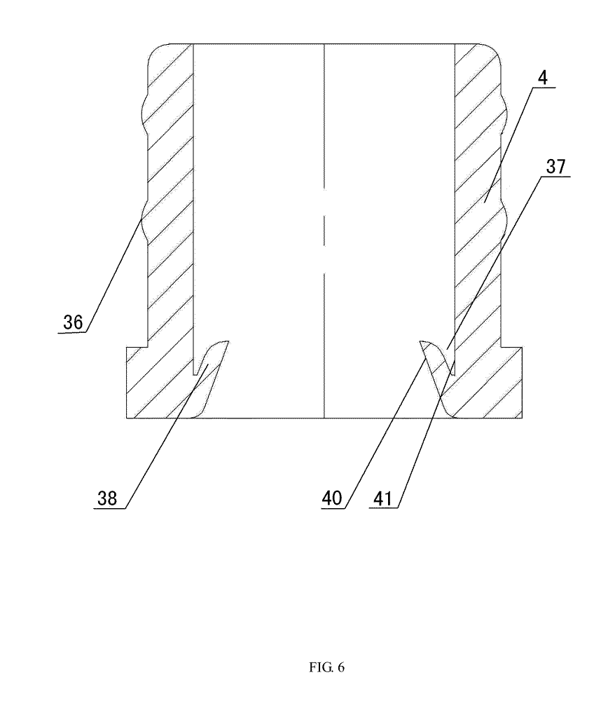

FIG. 7 is a schematic diagram of a barrel body of the present invention.

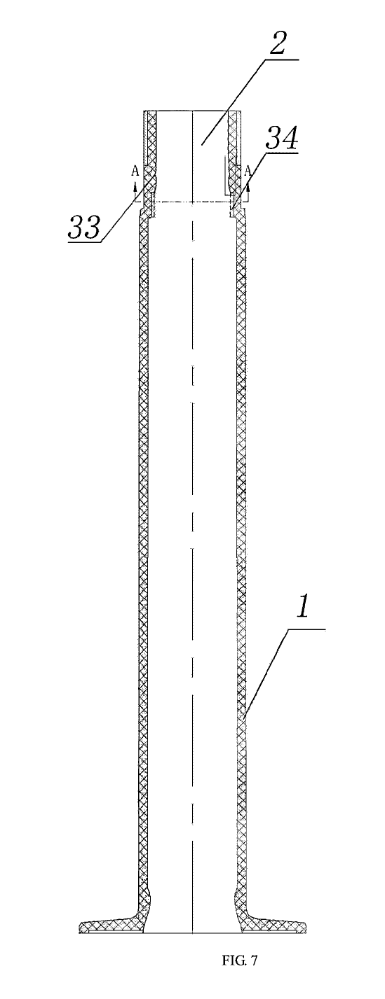

FIG. 8 is a cross-sectional view of FIG. 7 along A-A direction of the present invention.

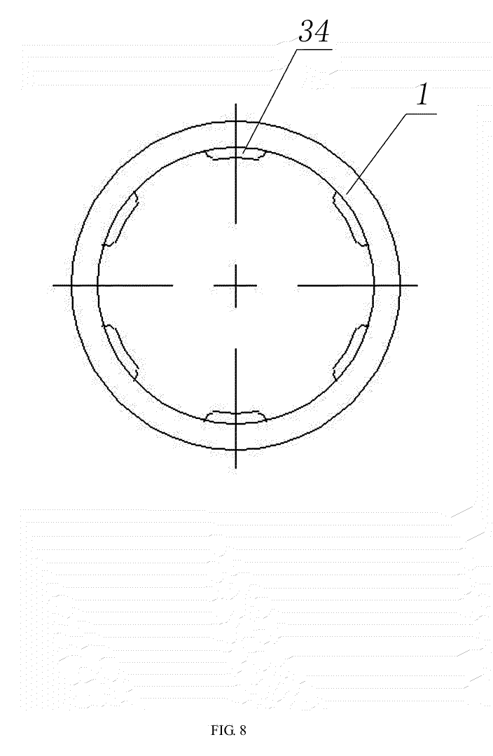

FIG. 9 is a schematic diagram of a plunger of the present invention.

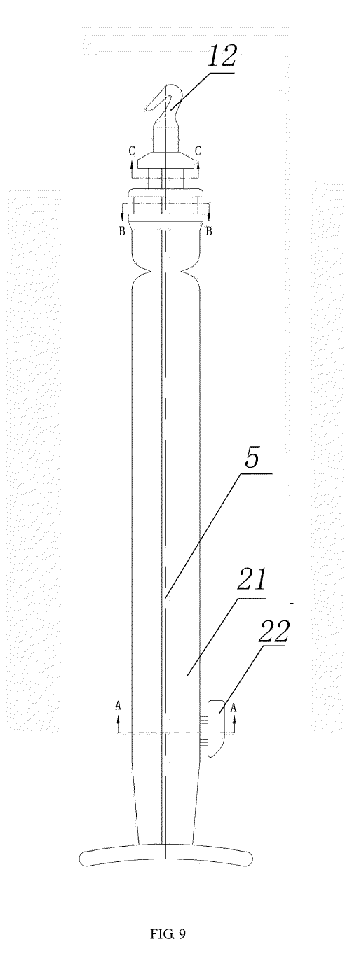

FIG. 10 is a schematic diagram of a hook body of the present invention.

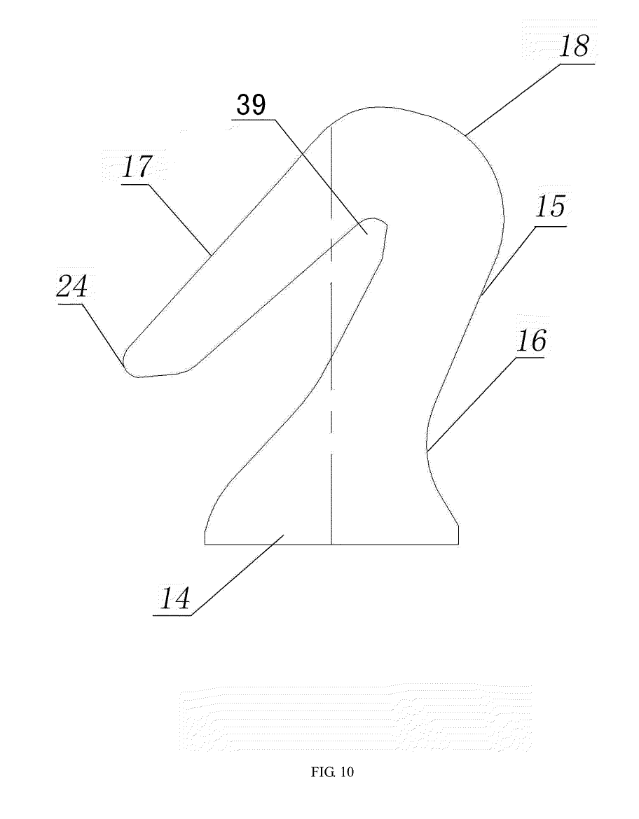

FIG. 11 is an schematic diagram of assembly of the needle base body and the internal sleeved retracting locking member of the present invention.

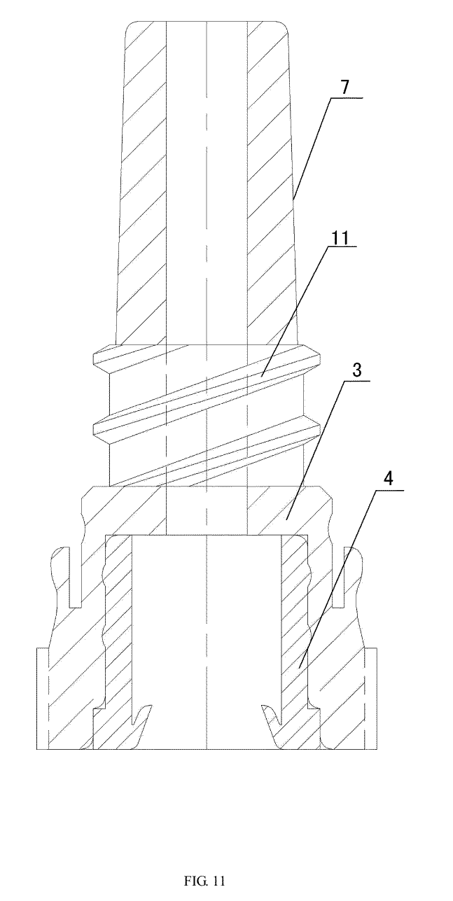

FIG. 12 is a view of a first use state of the present invention.

FIG. 13 is a view of a second use state of the present invention.

FIG. 14 is a view of a third use state of the present invention.

FIG. 15 is a view of a fourth use state of the present invention.

FIG. 16 is a view of a fifth use state of the present invention.

FIG. 17 is a view of a sixth use state of the present invention.

### DETAILED DESCRIPTION OF THE PREFERRED EMBODIMENT

Hereinafter, preferred embodiments of the present disclosure will be described in detail with reference to the accompanying drawings.

The present invention is a retractable self-destroying safety syringe with replaceable needle, which comprises a barrel body 1, a needle base body 3 disposed in the barrel neck 2 at the end of the barrel body 1, a plunger 5 disposed in the barrel body 1, and a rubber piston 6 disposed near the end of the plunger 5. It differs from current techniques in that: an elastic buffering engaging mechanism is formed between the barrel neck 2 and the needle base body 3, which can be disposed either on an outer wall of the needle base body 3 or on an inner wall of the barrel neck 2. The needle base body and the barrel body can be interlocked stably and released easily due to the elastic buffering engaging mechanism.

In an embodiment of the present invention, the elastic buffering engaging mechanism is disposed on the needle base body 3. The needle base body 3 has a hollow cavity in where an internal sleeved retracting locking member 4 is disposed, or an exterior of the needle base body 3 is sleeved to a sleeve-type retracting locking member. A replaceable needle assembly 7 is formed on an upper portion of the needle base body 3 and an elastic sealing engaging portion 8 is formed on a lower portion of the needle base body 3, wherein the replaceable needle assembly 7 and the elastic sealing engaging portion 8 are integrated together to form a single structure. An annular buffering gap 9 is defined by the surface of the elastic sealing engaging portion 8, a ring-shaped engagement part 10 is formed on an outer wall of the elastic sealing engaging portion 8 outside the annular buffering gap 9, anti-rotation ribs 13 are formed on the outer wall of the elastic sealing engaging portion 8, an external thread assembly 11 is formed on the replaceable needle assembly 7, and an asymmetrical elastic retracting locking member is disposed at the end of the plunger 5.

In another embodiment of the present invention, the elastic buffering engaging mechanism is disposed on the needle base body 3. The needle base body 3 has a hollow cavity in where an internal sleeved retracting locking member 4 is disposed, or an exterior of the needle base body 3 is sleeved to a sleeve-type retracting locking member. A replaceable needle assembly 7 is formed on an upper portion of the needle base body 3 and an elastic sealing engaging portion 8 is formed on a lower portion of the needle base body 3, wherein the replaceable needle assembly 7 and the elastic sealing engaging portion 8 are independent parts to form a split assembly structure. An annular buffering gap 9 is defined by a surface of the elastic sealing engaging portion 8, a ring-shaped engagement part 10 is formed on an outer wall of the elastic sealing engaging portion 8 outside the annular buffering gap 9, anti-rotation ribs 13 are formed on the outer wall of the elastic sealing engaging portion 8, an external thread assembly 11 is formed on the replaceable needle assembly 7, and an asymmetrical elastic retracting locking member is disposed on an end of the plunger 5.

In the detailed embodiment, the central axis of the asymmetrical elastic retracting locking member is on a symmetric central line of the plunger, an locking portion is respectively formed on both sides of the central axis and the locking portions on the both sides are asymmetrical to each other, wherein one of the locking portions on one side of the central axis is higher or lower than the other locking portion on the other side, and the width of one of the locking portions on one side of the central axis is larger or smaller than that of the other locking portion on the other side; an elastic mechanism is formed on the locking portion on one side or the locking portions on both sides, and a distance between an outer edge of the locking portion on one side and the symmetric central line of the plunger is ranged from 1 mm to 20 mm. Through the above structures, the elastic structure of all or part of the asymmetrical elastic retracting locking member can be achieved so that the asymmetrical elastic retracting locking member can enters the cavity of the needle base body smoothly and be locked with each other completely when retracted. An asymmetrical force can be applied on the needle base body though an asymmetrical locking structure so that the needle base body is deviated to one side after entering into the barrel body 1 for realizing the safety operation of the syringe.

In the detailed embodiment, the asymmetrical elastic retracting locking member is a hook 12. The hook 12 is made of plastic material, which is integrated together with the plunger 5 and is formed by one-time molding process. The uniformity of the materials can ensure the stability of the interlocking function of the plunger and the integral structure can reduce assembly processes, so as to improve production efficiency and reduce manufacturing cost.

In the detailed embodiment, a base portion 14 is formed at a lower end of the hook 12, a first arm 15 deflected to one side is formed on an upper part of the base portion 14, a concave curved angle 16 or a corner is defined by an outer sidewall of the junction between the first arm 15 and the base portion 14, and the concave curved angle 16 is ranged from 45 to 179 degrees. A lower end of the first arm 15 is connected with an upper end of the base portion 14, an upper end of the first arm 15 is connected with one end of a second arm 17 and another end of the second arm 17 is suspended. A curved surface 18 is formed on an outer side of the junction between the first arm 15 and the second arm 17, an inner angle 39 ranged from 2 to 90 degrees is defined by an inner side of the junction between the first arm 15 and the second arm 17. The width of an opening formed between the suspended end of the second arm 17 and the first arm 15 is ranged from 0.5 mm to 40 mm, the length of the first arm 15 is ranged from 0.5 mm to 50 mm, and the length of the second arm 17 is ranged from 0.5 mm to 60 mm. A tip 24 is formed on the suspended end of the second arm 17, and the suspended end of the second arm 17 protrudes beyond an outer edge of the base portion 14.

In the detailed embodiment, four reinforcing ribs 21 arranged in a cross shape and at equal angles are formed on the plunger 5, with a pre-stopper piece 22 disposed on an outer edge of the reinforcing ribs 21. The pre-stopper piece 22 is connected with the reinforcing ribs 21 by at least two connection portions 23. After the syringe is assembled, a top end of the pre-stopper piece 22 is just against on the lower end of the barrel body thereby preventing the plunger from moving forwardly and the syringe from being wasted when the syringe is under an external force before use. In use, the pre-stopper piece 22 can be removed from the reinforcing ribs by simply bending along the connection portions.

In the detailed embodiment, a replaceable needle base 24 is connected with the external thread assembly 11 on the replaceable needle assembly 7, a needle tube 25 is disposed on the center of an upper part of the replaceable needle base 24, a columnar cavity 30 interconnected with the needle tube 25 is formed in the replaceable needle base 24. An internal thread assembly 31 interlocked with the external thread assembly 11 is formed on an inner wall of the cavity of the replaceable needle base 24; an upper portion of the columnar cavity 30 is a truncated cone, and the taper of the truncated cone is 6:100. The taper is an international general parameter which can ensure the sealing performance of the assembly.

In the detailed embodiment, anti-rotation ribs 13 are distributed evenly in an annular shape on the outer wall of the elastic sealing engaging portion 8, and the ends of the anti-rotation ribs 13 are semicircles or cones.

In the detailed embodiment, an upper portion of the replaceable needle assembly 7 is a truncated cone, the taper of the truncated cone is 6:100, and the external thread assembly 11 is formed on an outer wall of a lower portion of the replaceable needle assembly 7. The taper is an international general parameter which can ensure the sealing performance of the assembly.

In the detailed embodiment, an annular step 26 or an annular slope is formed on the transition between the replaceable needle assembly 7 and the elastic sealing engaging portion 8.

In the detailed embodiment, the annular buffering gap 9 is an integrated annular groove-like structure or a non-integrated annular with segmental structures. The height from an opening of the groove to a bottom thereof is ranged from 0.05 mm to 4 mm, the width of the annular buffering gap 9 is ranged from 0.05 mm to 4 mm, the distance from an outer groove wall of the annular buffering gap 9 to a top of a curved surface of the ring-shaped engagement part 10 is ranged from 0.1 mm to 6 mm, the distance from the opening of the outer groove wall of the annular buffering gap 9 to the outer wall of the ring-shaped engagement part 10 is ranged from 0.1 mm to 6 mm, and the distance from the bottom of the outer groove wall of the annular buffering gap 9 to the outer wall of the ring-shaped engagement part 10 is ranged from 0.1 mm to 6 mm. The surface of the ring-shaped engagement part 10 is a curved surface, and the distance from the top of the curved surface of the ring-shaped engagement part 10 to an upper edge of the curved surface is less than the distance from the top of the curved surface of the ring-shaped engagement part 10 to a lower edge of the curved surface. An annular recess 27 is formed on the outer wall of the elastic sealing engaging portion 8 below the ring-shaped engagement part 10.

In the detailed embodiment, the annular recess 27 is in the same horizontal plane as a groove bottom of the annular buffering gap 9, or higher than a groove bottom of the annular buffering gap 9.

In the detailed embodiment, an O-ring assembly groove 28 is formed on an outer wall of the elastic sealing engaging portion 8 below the annular step 26, and an O-ring 29 is assembled into the O-ring assembly groove 28.

In the detailed embodiment, the exterior of the barrel neck 2 of the barrel body 1 is covered by a barrel body cap 32 for protecting the end of the barrel body and the end of the needle base body 3.

In the detailed embodiment, an annular engagement sealing recess 33 matching the ring-shaped engagement part 10 is disposed on an inner wall of the barrel neck 2 of the barrel body 1. The depth of the annular engagement sealing recess 33 is ranged from 0.01 mm to 4 mm and the height of the annular engagement sealing recess 33 is ranged from 0.01 mm to 6 mm. The recess bottom of the annular engagement sealing recess 33 is a curved surface, and anti-rotation ribs 34 matching the anti-rotation ribs 13 are formed on the inner wall of the barrel neck 2 below the annular engagement sealing recess 33.

In the detailed embodiment, at least one annular stopping groove of the needle base 35 is formed on the inner wall of the cavity of the needle base body 3, the internal sleeved retracting locking member 4 is disposed in the cavity of the needle base body 3, and at least one stopping flange ring 36 matching the annular stopping groove of the needle base 35 is formed on an outer wall of the internal sleeved retracting locking member 4.

In the detailed embodiment, the internal sleeved retracting locking member 4 has a hollow inner cavity, a ring-shaped engaging and locking portion 38 is formed on the inner wall of the cavity near an opening of a lower end of internal sleeved retracting locking member 4. The ring-shaped engaging and locking portion has a certain damping elastic function. When the asymmetrical elastic retracting locking member contacts the ring-shaped engaging and locking portion during the pushing process, elastic buffering deformation occurs to both of them under interactive forces, so that the asymmetrical elastic retracting locking member can smoothly go through the ring-shaped engaging and locking portion in the pushing process.

In the detailed embodiment, an annular recess 37 is formed on an upper part of the ring-shaped engaging and locking portion 38. The thickness of an inner sidewall 40 of the annular recess 37 is ranged from 0.01 mm to 3 mm, an angle ranged from 2 to 90 degrees is formed between the inner side wall of the annular recess 37 and an outer sidewall 41 of the annular recess 37, and the inner diameter of an upper end of the inner side wall of the annular recess 37 is ranged from 1 mm to 80 mm. The above mentioned technical parameters ensure the elastic buffering function of the ring-shaped engaging and locking portion 38, which is realized especially at the outer wall 41. In the detailed embodiment, the width of an opening of the annular recess 37 is greater than the width of a bottom thereof.

After the asymmetrical elastic retracting locking member slides into the internal sleeved retracting locking member 4, one side end of the asymmetrical elastic retracting locking member (the suspended end of the second arm) is just blocked into the annular groove 37 when retracting the plunger, so that the end of the plunger is completely engaged with the internal sleeved retracting locking member 4.

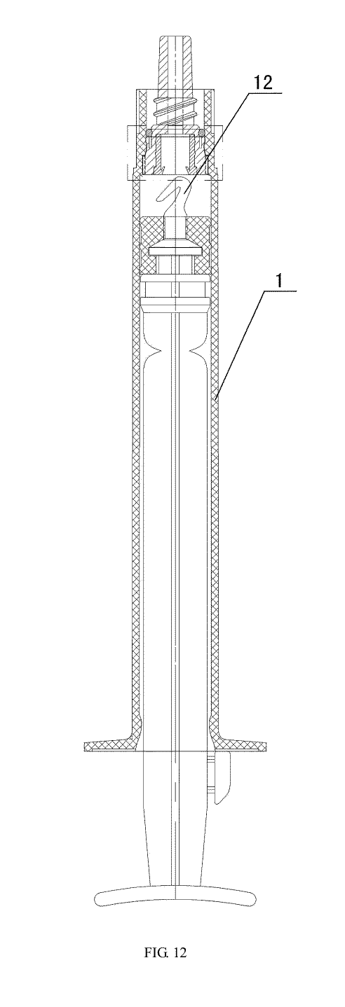

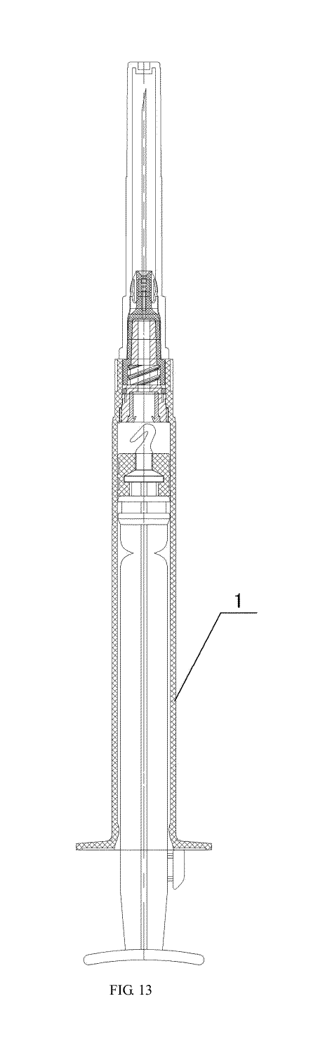

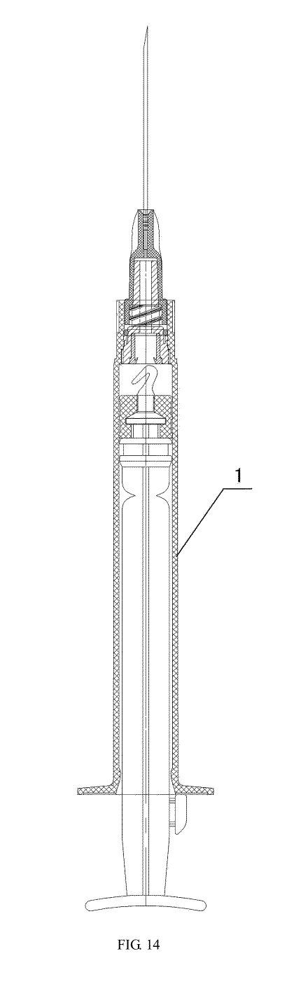

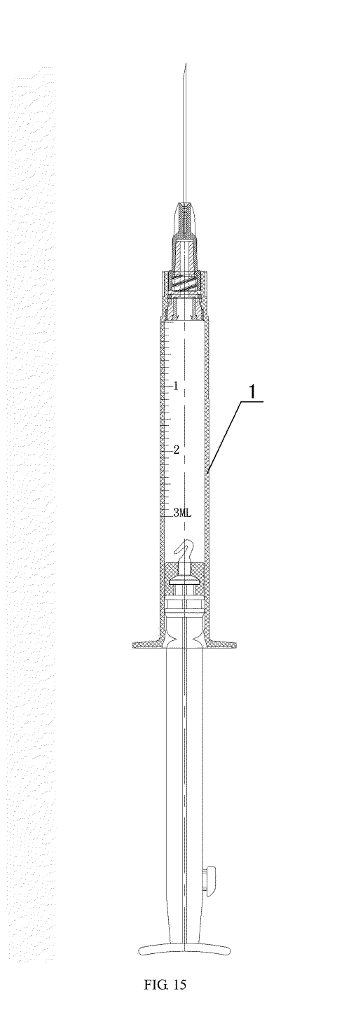

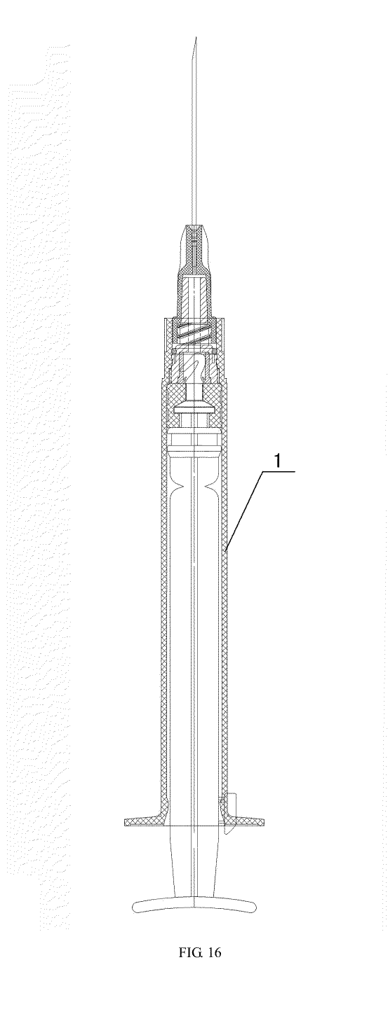

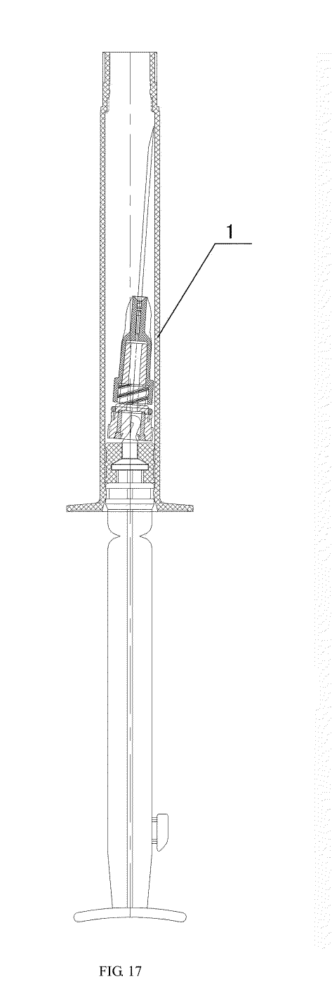

In the practical operation, as shown in FIG. 12 to FIG. 17, the replaceable needle base is rotated into the end of the needle base body. It is realized that both of them are assembled tightly because of the cooperation between the tread structure and the structure with taper 6:100. When the injection begins after drawing the liquid, as slowly pushing the plunger and then contact occurs between the asymmetrical elastic retracting locking member and the internal sleeved retracting locking member, one side of the asymmetrical elastic retracting locking member is deformed so that the width is narrowed and then the asymmetrical elastic retracting locking member enters the cavity of the internal sleeved retracting locking member due to the greater total width of the asymmetrical elastic retracting locking member than the inner diameter of the annular groove 37 in the internal sleeved retracting locking member. After the asymmetrical elastic retracting locking member enters the cavity, the force is removed and the asymmetrical elastic retracting locking member restores to its original state. Pull the plunger backwards when the injection is finished, and the side end of the asymmetrical elastic retracting locking member (the suspended end of the second arm of the hook) is just stuck in the annular groove of the internal sleeved retracting locking member. The annular buffering gap of the elastic sealing engaging portion on the needle base body is deformed under the pulling force of the plunger that is transferred to the needle base body through the internal sleeved retracting locking member. The ring-shaped engagement part slides out from the annular engagement sealing recess of the barrel body as the annular buffering gap is being narrowed down. Finally, the whole needle is pulled into the cavity of the barrel body. The needle is deflected to one side and deviated from the regular displacement track under the force generated from the asymmetrical elastic retracting locking member, so that the needle cannot be re-assembled through the barrel neck, thereby realizing safe operation and self-destruction of the syringe.

The above content describes the present invention in combination with the detailed preferred embodiment method. However, it should not be considered that the detailed embodiment of the present invention is only limited to the above description. A person of ordinary skill in the art, without departing from the concept of the present invention, may make many simple derivations or alternations, which should be deemed within the scope of the present invention.

1. A retractable self-destroying safety syringe with a replaceable needle, comprising a barrel body (1), a needle base body (3) disposed in a barrel neck (2) at an end of the barrel body (1), a plunger (5) disposed in the barrel body (1), and a rubber piston (6) disposed near an end of the plunger (5), characterized in that: an elastic buffering engaging mechanism is formed between the barrel neck (2) and the needle base body (3).

2. The retractable self-destroying safety syringe with replaceable needle as claimed in claim 1, characterized in that: the elastic buffering engaging mechanism is disposed on the needle base body (3) having a hollow cavity in where an internal sleeved retracting locking member (4) is disposed, or an exterior of the needle base body (3) is sleeved to a sleeve-type retracting locking member; a replaceable needle assembly (7) is formed on an upper portion of the needle base body (3) and an elastic sealing engaging portion (8) is formed on a lower portion of the needle base body (3), wherein the replaceable needle assembly (7) and the elastic sealing engaging portion (8) are integrated together to form a single structure; an annular buffering gap (9) is defined by a surface of the elastic sealing engaging portion (8); a ring-shaped engagement part (10) is formed on an outer wall of the elastic sealing engaging portion (8) outside the annular buffering gap (9); anti-rotation ribs (13) are formed on the outer wall of the elastic sealing engaging portion (8); an external thread assembly; (11) is formed on the replaceable needle assembly (7), and an asymmetrical elastic retracting locking member is disposed at an end of the plunger (5).

3. The retractable self-destroying safety syringe with replaceable needle as claimed in claim 1, characterized in that: the elastic buffering engaging mechanism is disposed on the needle base body (3) having a hollow cavity in where an internal sleeved retracting locking member (4) is disposed, or an exterior of the needle base body (3) is sleeved to a sleeve-type retracting locking member; a replaceable needle assembly (7) is formed on an upper portion of the needle base body (3) and an elastic sealing engaging portion (8) is formed on a lower portion of the needle base body (3), wherein the replaceable needle assembly (7) and the elastic sealing engaging portion (8) are independent parts to form a split assembly structure; an annular buffering gap (9) is defined by a surface of the elastic sealing engaging portion (8); a ring-shaped engagement part (10) is formed on an outer wall of the elastic sealing engaging portion (8) outside the annular buffering gap (9); anti-rotation ribs (13) are formed on the outer wall of the elastic sealing engaging portion (8); an external thread assembly (11) is formed on the replaceable needle assembly (7), and an asymmetrical elastic retracting locking member is disposed on an end of the plunger (5).

4. The retractable self-destroying safety syringe with replaceable needle as claimed in claim 2, characterized in that: a central axis of the asymmetrical elastic retracting locking member is on a symmetric central line of the plunger, an locking portion is respectively formed on both sides of the central axis and the locking portions on the both sides are asymmetrical to each other, wherein one of the locking portions on one side of the central axis is higher or lower than the other locking portion on the other side; and the width of one of the locking portions on one side of the central axis is larger or smaller than the width of the other locking portion on the other side; an elastic mechanism is formed on the locking portion on one side or the locking portions on both sides, and a distance between an outer edge of the locking portion on one side and the symmetric central line of the plunger is ranged from 1 mm- to 20 mm.

5. The retractable self-destroying safety syringe with replaceable needle as claim in claim 2, characterized in that: the asymmetrical elastic retracting locking member is a hook (12) made of plastic material, and the hook is integrated together with the plunger (5) and is formed by one-time molding process.

6. The retractable self-destroying safety syringe with replaceable needle as claimed in claim 5, characterized in that: a base portion (14) is formed at a lower end of the hook (12); a first arm (15) deflected to one side is formed on an upper part of the base portion (14); a concave curved angle (16) or a corner is defined by an outer sidewall of a junction between the first arm (15) and the base portion (14), and the concave curved angle (16) is ranged from 45 to 179 degrees; a lower end of the first arm (15) is connected with an upper end of the base portion (14), an upper end of the first arm (15) is connected with one end of a second arm (17) and another end of the second arm (17) is suspended; a curved surface (18) is formed on an outer side of a junction between the first arm (15) and the second arm (17), an inner angle (39) ranged from 2- to 90 degrees is defined by an inner side of the junction between the first arm (15) and the second arm (17); a width of an opening formed between the suspended end of the second arm (17) and the first arm (15) is ranged from 0.5 mm- to 40 mm; the length of the first arm (15) is ranged from 0.5 mm to 50 mm, and the length of the second arm (17) is ranged from 0.5 mm- to 60 mm.

7. The retractable self-destroying safety syringe with replaceable needle as claimed in claim 1, characterized in that: four reinforcing ribs (21) arranged in a cross shape and at equal angles are formed on the plunger (5), with a pre-stopper piece (22) disposed on an outer edge of the reinforcing ribs (21).

8. The retractable self-destroying safety syringe with replaceable needle as claimed in claim 7, characterized in that: the pre-stopper piece (22) is connected with the reinforcing ribs (21) by at least two connection portions (23).

9. The retractable self-destroying safety syringe with replaceable needle as claimed in claim 6, characterized in that: a tip (24) is formed on the suspended end of the second arm (17), and the suspended end of the second arm (17) protrudes beyond an outer edge of the base portion (14).

10. The retractable self-destroying safety syringe with replaceable needle as claimed in claim 2, characterized in that: a replaceable needle base (24) is connected with the external thread assembly (11) on the replaceable needle assembly (7), a needle tube (25) is disposed on a center of an upper part of the replaceable needle base (24); a columnar cavity (30) interconnected with the needle tube (25) is formed in the replaceable needle base (24), an internal thread assembly (31) interlocked with the external thread assembly (11) is formed on an inner wall of the cavity of the replaceable needle base (24), an upper portion of the columnar cavity (30) is a truncated cone, and the taper of the truncated cone is 6:100.

11. The retractable self-destroying safety syringe with replaceable needle as claimed in claim 2, characterized in that: anti-rotation ribs (13) are distributed evenly in an annular shape on the outer wall of the elastic sealing engaging portion (8), and the ends of the anti-rotation ribs (13) are semicircles or cones.

12. The retractable self-destroying safety syringe with replaceable needle as claimed in claim 2, characterized in that: an upper portion of the replaceable needle assembly (7) is a truncated cone, the taper of the truncated cone is 6:100, and the external thread assembly (11) is formed on an outer wall of a lower portion of the replaceable needle assembly (7).

13. The retractable self-destroying safety syringe with replaceable needle as claimed in claim 2, characterized in that: an annular step (26) or an annular slope is formed on a transition between the replaceable needle assembly (7) and the elastic sealing engaging portion (8).

14. The retractable self-destroying safety syringe with replaceable needle as claim in claim 2, characterized in that: the annular buffering gap (9) is an annular groove-like structure, a height from an opening of the groove to a bottom thereof is ranged from 0.05 mm- to 4 mm, a width of the annular buffering gap (9) is ranged from 0.05 mm- to 4 mm, the distance from an outer groove wall of the annular buffering gap (9) to a top of a curved surface of the ring-shaped engagement part (10) is ranged from 0.1 mm- to 6 mm, the distance from the opening of the outer groove wall of the annular buffering gap (9) to the outer wall of the ring-shaped engagement part (10) is ranged from 0.1 mm- to 6 mm, the distance from the bottom of the outer groove wall of the annular buffering gap (9) to the outer wall of the ring-shaped engagement part (10) is ranged from 0.1 mm- to 6 mm; a surface of the ring-shaped engagement part (10) is a curved surface, and the distance from the top of the curved surface of the ring-shaped engagement part (10) to an upper edge of the curved surface is less than the distance from the top of the curved surface of the ring-shaped engagement part (10) to a lower edge of the curved surface, and an annular recess (27) is formed on the outer wall of the elastic sealing engaging portion (8) below the ring-shaped engagement part (10).

15. The retractable self-destroying safety syringe with replaceable needle as claimed in claim 14, characterized in that: the annular recess (27) is in the same horizontal plane as a groove bottom of the annular buffering gap (9), or higher than a groove bottom of the annular buffering gap (9).

16. The retractable self-destroying safety syringe with replaceable needle according to claim 13, characterized in that: an O-ring assembly groove (28) is formed on an outer wall of the elastic sealing engaging portion (8) below the annular step (26), and an O-ring (29) is assembled into the O-ring assembly groove (28).

17. The retractable self-destroying safety syringe with replaceable needle according to claim 1, characterized in that: the exterior of the barrel neck (2) of the barrel body (1) is covered by a barrel body cap (32).

18. The retractable self-destroying safety syringe with replaceable needle as claimed in claim 1, characterized in that: an annular engagement sealing recess (33) matching the ring-shaped engagement part (10) is disposed on an inner wall of the barrel neck (2) of the barrel body (1), the depth of the annular engagement sealing recess (33) is ranged from 0.01 mm- to 4 mm and the height of the annular engagement sealing recess (33) is ranged from 0.01 mm- to 6 mm, and a recess bottom of the annular engagement sealing recess (33) is a curved surface, and anti-rotation ribs (34) matching the anti-rotation ribs (13) are formed on the inner wall of the barrel neck (2) below the annular engagement sealing recess (33).

19. The retractable self-destroying safety syringe with replaceable needle as claimed in claim 1, characterized in that: at least one annular stopping groove of the needle base (35) is formed on the inner wall of the cavity of the needle base body (3), the internal sleeved retracting locking member (4) is disposed in the cavity of the needle base body (3), and at least one stopping flange ring (36) matching the annular stopping groove inside the needle base (35) is formed on an outer wall of the internal sleeved retracting locking member (4).

20. The retractable self-destroying safety syringe with replaceable needle as claimed in claim 2, characterized in that: the internal sleeved retracting locking member (4) has a hollow inner cavity, ring-shaped engaging and locking portion (38) is formed on the inner wall of the cavity near an opening of a lower end of the internal sleeved retracting locking member (4).

21. The retractable self-destroying safety syringe with replaceable needle as claimed in claim 20, characterized in that: an annular recess (37) is formed on an upper part of the ring-shaped engaging and locking portion (38), a thickness of an inner sidewall (40) of the annular recess (37) is ranged from 0.01 mm- to 3 mm, an angle ranged from 2- to 90 degrees is formed between the inner side wall of the annular recess (37) and an outer sidewall (41) of the annular recess (37), and an inner diameter of an upper end of the inner side wall of the annular recess (37) is ranged from 1 mm- to 80 mm.

22. The retractable self-destroying safety syringe with replaceable needle as claimed in claim 21, characterized in that: the width of an opening of the annular recess (37) is greater than the width of a bottom thereof.
